(19) United States
(12) Patent Application Publication
(10) Pub. No.: US 2025/0359893 A1
(43) Pub. Date: Nov. 27, 2025

(54) Intraosseous Access Systems and Methods

(71) Applicant: Bard Access Systems, Inc., Salt Lake City, UT (US)

(72) Inventors: Ravi Prakash, Bengaluru (IN); Mukilan Balamurugan, Theni (IN); Deivakumaran Rajendran, Bengaluru (IN); Shawn Ray Isaacson, Layton, UT (US)

(21) Appl. No.: 18/674,679

(22) Filed: May 24, 2024

(51) Int. Cl. A61B 17/34; A61B 17/16; A61M 39/02

(52) U.S. Cl. CPC A61B 17/3472; A61B 17/1628; A61M 39/02

(57) ABSTRACT

An intraosseous access system includes an intraosseous driver having socket configured to receive a shaft of an obturator assembly. An O-ring disposed within a groove of the shaft inhibits removal of the shaft from the socket. The O-ring can be compressed within the groove to define a frictional force between the socket and the shaft. The socket can include recesses and the O-ring may expand into the recesses to inhibit removal of the shaft from the socket. The driver can include a latch mechanism configured to selectively prevent removal of the shaft and allow removal of the shaft. An adapter can be positioned between the driver and obturator assembly where the adapter includes the latch mechanism. The latch mechanism include an actuator to selectively transition the latch mechanism between a retaining state and a releasing state.

### BACKGROUND

Many devices, systems, and methods have been developed to for accessing an interior of a bone of a patient, including for such purposes as intraosseous access. Known devices, systems, and methods, however, suffer from one or more drawbacks that can be resolved, remedied, ameliorated, or avoided by certain embodiments described herein.

### SUMMARY

Placing an intraosseous (“I.O.”) device requires driving a needle and an obturator of an access assembly through the patient's skin and tissue until the needle tip is pressed against a surface of the bone cortex. Exemplary bones which can be accessed include the proximal tibia or humerus. The needle is then drilled, using a power driver (e.g. electrical or spring), manual awl, or the like, through the outer cortex of the bone until the tip enters the medullary cavity. The user then ceases drilling, and the obturator is removed leaving the hollow needle in place. The proximal needle hub is accessed and fluids can be introduced.

Briefly summarized, disclosed herein is an intraosseous access system that, according to some embodiments, includes an intraosseous driver having a driver coupling interface and an obturator assembly including an obturator coupling interface coupled with the driver coupling interface such that operation of the intraosseous driver causes rotation of the obturator assembly. The obturator coupling interface includes a (i) shaft configured for insertion into a socket of the driver coupling interface so as to cause co-rotation of the shaft and the socket and (ii) an annular groove extending around the shaft, where the annual groove is utilized to inhibit decoupling of the obturator coupling interface from the driver coupling interface.

According to some embodiments of the intraosseous access system disclosed herein, the shaft includes a polygonal shape including a number of shaft sides and a corresponding number of shaft corners, where the shaft corners define a major diameter of the shaft, and the shaft sides define a minor diameter of the shaft that is less than the major diameter of the shaft. The shaft further includes a groove diameter that is less than the minor diameter of the shaft.

According to some embodiments of the intraosseous access system disclosed herein, the socket has a polygonal shape that corresponds to the polygonal shape of the shaft. Socket corners of the polygonal shape of the socket define a major diameter of the socket, and socket sides of the polygonal shape of the socket define a minor diameter of the socket that is less than the major diameter of the socket.

According to some embodiments of the intraosseous access system disclosed herein, the obturator coupling interface further comprises a retaining member disposed within the groove, where the retaining member defines, in a free state, a hoop shape having an outside diameter and an inside diameter.

According to some embodiments of the intraosseous access system disclosed herein, the inside diameter is less than the minor diameter of the shaft and the outside diameter is greater than the minor diameter of the shaft. According to further embodiments of the intraosseous access system disclosed herein, the outside diameter is less than the major diameter of the shaft.

According to some embodiments of the intraosseous access system disclosed herein, the outside diameter is greater than the minor diameter of the socket such that, during insertion of the shaft into the socket, the socket sides deflect the retaining member radially inward. According to some embodiments of the intraosseous access system disclosed herein, the outside diameter is less than the major diameter of the socket.

According to some embodiments of the intraosseous access system disclosed herein, the retaining member defines a frictional force with the socket sides, the frictional force inhibiting separation of the shaft from the socket.

According to some embodiments of the intraosseous access system disclosed herein, the minor diameter of the socket, the groove diameter, the outside diameter and the inside diameter are sized to define an interference fit of the retaining member between the socket sides and a bottom surface of the groove, the interference fit defining the frictional force.

According to some embodiments of the intraosseous access system disclosed herein, at least a subset of the socket sides includes a recess configured to receive a portion of the retaining member therein so as to inhibit separation of the shaft from the socket.

According to some embodiments of the intraosseous access system disclosed herein, the retaining member extends continuously and entirely around the hoop shape. According to further embodiments of the intraosseous access system disclosed herein, the retaining member includes an O-ring formed of a plastic or an elastomeric material.

According to some embodiments of the intraosseous access system disclosed herein, the driver coupling interface includes a latch mechanism configured to transition between (i) a retaining state, where separation of the obturator coupling interface from the driver coupling interface is prevented, and (ii) a releasing state, where separation of the obturator coupling interface from the driver coupling interface is allowed.

According to some embodiments of the intraosseous access system disclosed herein, the latch mechanism includes an actuation member coupled with a groove engagement member such that displacing the actuation member extracts the groove engagement member from the groove to transition the latch mechanism from the retaining state to the releasing state.

According to some embodiments of the intraosseous access system disclosed herein, the intraosseous access system further includes an adapter configured for placement between the obturator assembly and intraosseous driver, where the adapter includes an adapter shaft configured for insertion into the socket of the intraosseous driver and an adapter socket configured to receive the shaft of the obturator assembly.

Also disclosed herein is an adapter for an intraosseous access system that, according to some embodiments disclosed herein includes an adapter frame having (i) an adapter socket configured to receive a shaft of an obturator assembly, (ii) an adapter shaft configured for insertion into a socket of an intraosseous driver, and (iii) a latch mechanism that includes a groove engagement member configured for selective displacement into and out of a groove of the shaft of the obturator assembly.

Also disclosed herein is a method of accessing a medullary cavity that, according to some embodiments disclosed herein includes (i) coupling an obturator assembly to an intraosseous driver, where coupling the obturator assembly to the intraosseous driver includes utilizes a groove of a shaft of the obturator assembly to inhibit removal of the shaft from within a socket of the intraosseous driver; and (ii) operating the intraosseous driver that includes rotating the obturator assembly to penetrate a bone cortex with a distal tip of a needle of the obturator assembly.

According to some embodiments of the method disclosed herein, utilizing the groove of the shaft includes compressing a hoop shaped retaining member within the groove.

According to some embodiments of the method disclosed herein, the intraosseous driver includes a latch mechanism including a groove engagement member configured for selective displacement into and out of the groove, and coupling the obturator assembly to the intraosseous driver includes displacing the groove engagement member into the groove of the shaft to prevent removal of the shaft from within the socket of the intraosseous driver.

According to some embodiments of the method disclosed herein, the latch mechanism includes an actuator coupled with the groove engagement member, and the method further includes displacing the actuator to extract the groove engagement member from the groove to enable removal of the shaft from within the socket of the intraosseous driver.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates an exploded view of an embodiment of an intraosseous access system, in accordance with embodiments disclosed herein.

FIG. 1B is a perspective view of an embodiment of an obturator assembly of the intraosseous access system of FIG. 1A, in accordance with embodiments disclosed herein.

FIG. 1C is a further perspective view of the obturator assembly of FIG. 1A, in accordance with embodiments disclosed herein.

FIG. 1D illustrates a side view of a portion of the obturator assembly including a shaft and a groove in the shaft, in accordance with embodiments disclosed herein.

FIG. 1E illustrates detailed cross-sectional view of the shaft of FIG. 1D cut along the sectioning line 1E-1E, in accordance with embodiments disclosed herein.

FIG. 1F illustrates a detailed end view of a socket of the driver of the intraosseous access system of FIG. 1A, in accordance with embodiments disclosed herein.

FIG. 1G illustrates a detailed perspective view of the socket of FIG. 1F, in accordance with embodiments disclosed herein.

FIG. 1H illustrates a cross-sectional side view of a portion of the intraosseous access system of FIG. 1A in an assembled state, in accordance with embodiments disclosed herein.

FIG. 2A illustrates an exploded view of a portion of a second embodiment of an intraosseous access system, in accordance with embodiments disclosed herein.

FIG. 2B illustrates cross-sectional perspective side view of the portion intraosseous access system of FIG. 2A, in accordance with embodiments disclosed herein.

FIG. 2C illustrates detailed cross-sectional side view of portion intraosseous access system of FIG. 2A in an assembled state including a latch mechanism in a retaining state, in accordance with embodiments disclosed herein.

FIG. 2D illustrates detailed cross-sectional side view of FIG. 2C with the latch mechanism in a releasing state, in accordance with embodiments disclosed herein.

FIG. 3 illustrates an exploded view of a portion of a third embodiment of an intraosseous access system including an adapter, in accordance with embodiments disclosed herein.

FIG. 4 is block diagram of a method of accessing a medullary cavity, in accordance with embodiments disclosed herein.

FIG. 5 illustrates an exploded view of a portion of a fourth embodiment of an intraosseous access system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

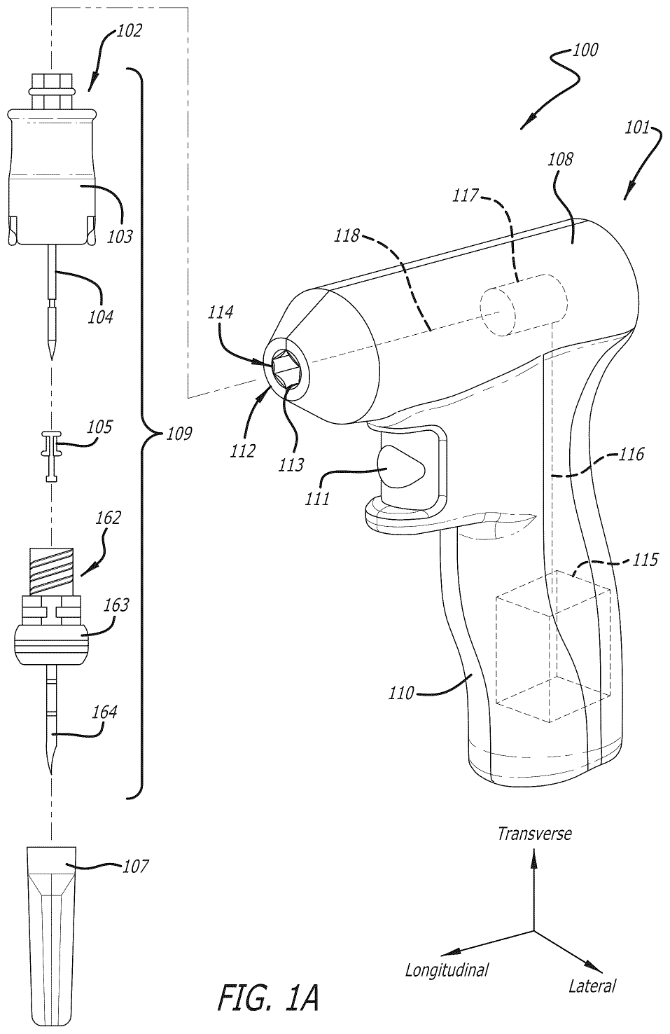

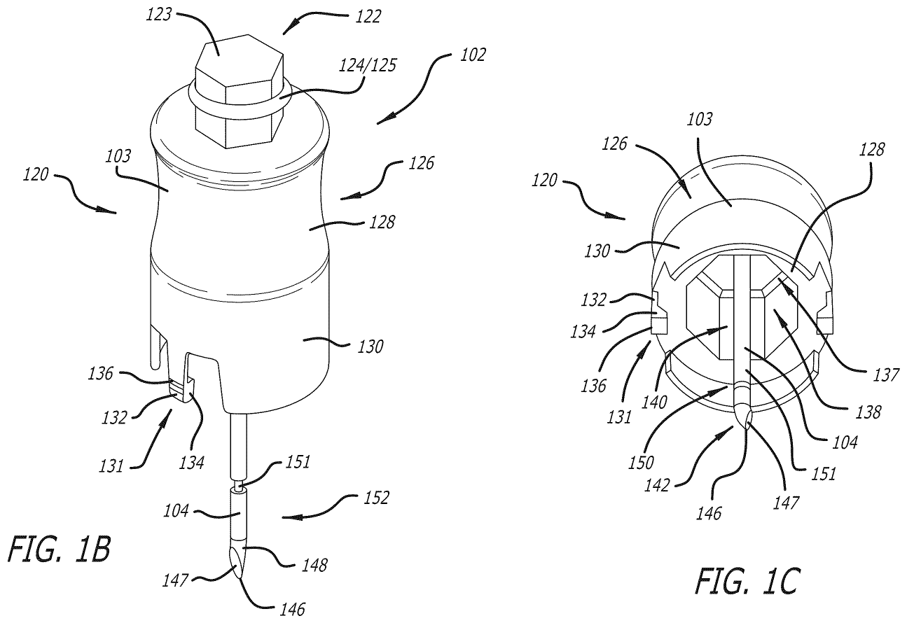

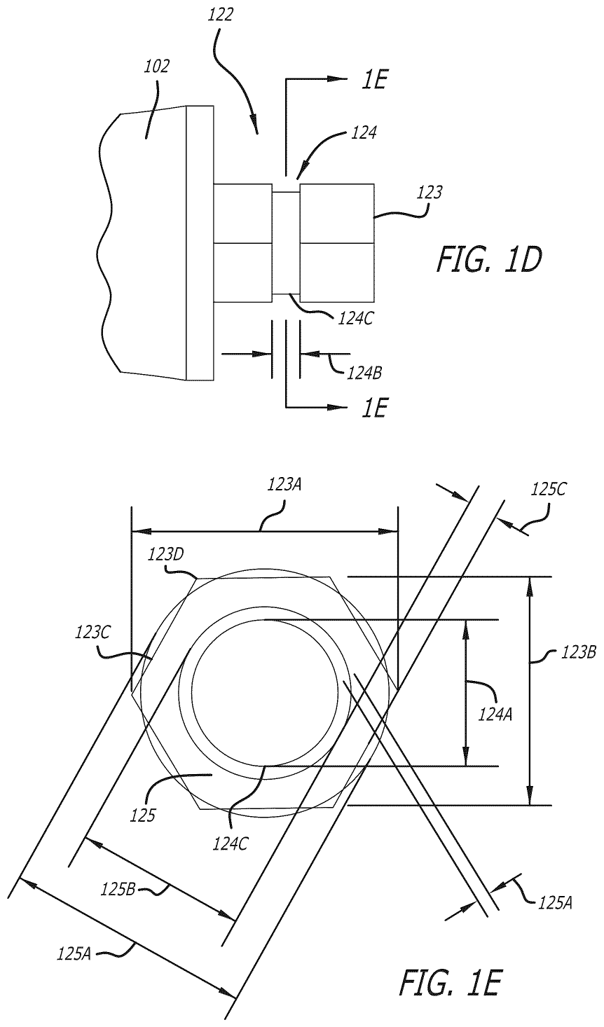

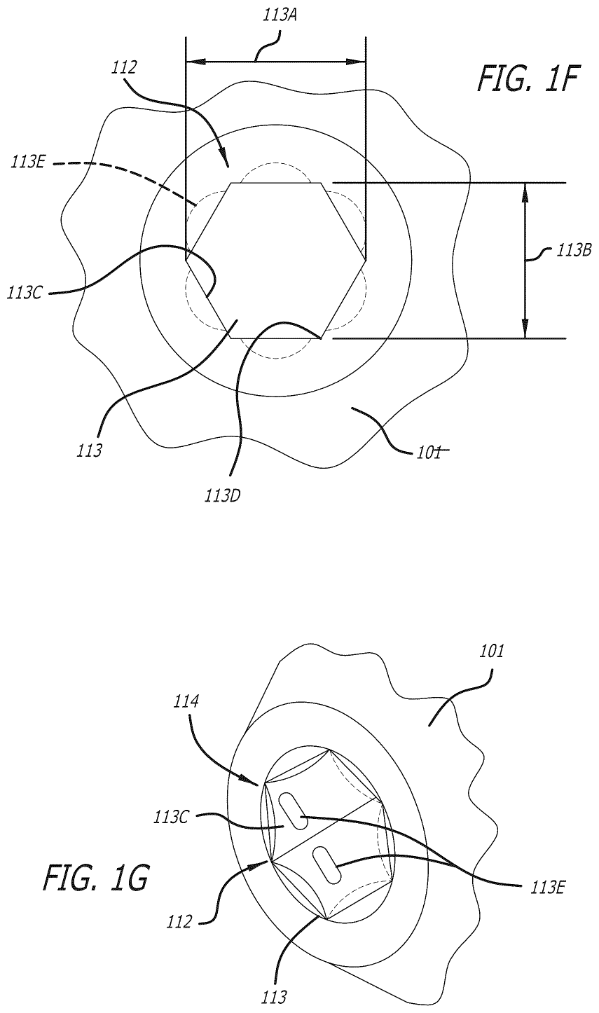

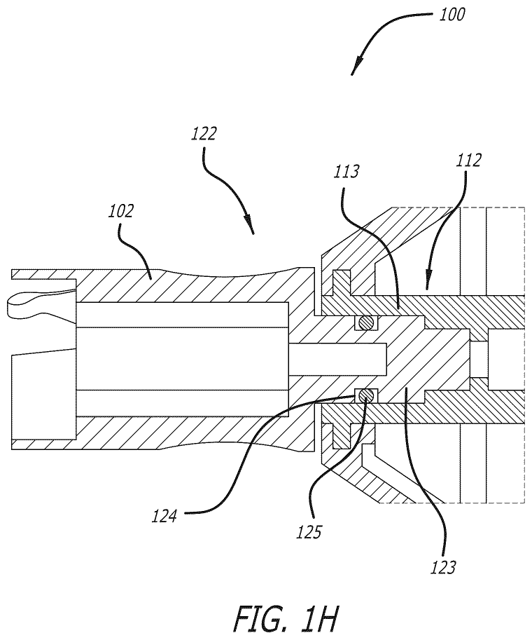

As shown in FIG. 1A, and to assist in the description of embodiments described herein, a longitudinal axis extends substantially parallel to an axial length of a needle 164 extending from the driver 101. A lateral axis extends normal to the longitudinal axis, and a transverse axis extends normal to both the longitudinal and lateral axes.

The present disclosure relates generally to intraosseous (“I.O.”) access devices, systems, and methods thereof. FIG. 1A shows an exploded view of an exemplary embodiment of an intraosseous access system 100, with some components thereof shown in elevation and another shown in perspective. The intraosseous access system 100 can be used to penetrate skin and underlying hard bone for intraosseous access, such as, for example to access the marrow of the bone and/or a vasculature of the patient via a pathway through an interior of the bone.

In an embodiment, the system includes a driver 101 and an access assembly 109. The driver 101 can be used to rotate the access assembly 109 into a bone of a patient. In embodiments, the driver 101 can be automated or manual. In an embodiment, the driver 101 is an automated driver 108. For example, the automated driver 108 can be a drill that achieves high rotational speeds.

The intraosseous access system 100 can further include an obturator assembly 102, and a needle assembly 162, which may be referred to, collectively, as the access assembly 109. The access assembly 109 may also be referred to as an access system. The obturator assembly 102 is referred to as such herein for convenience. In an embodiment, the obturator assembly 102 includes an obturator 104. However, in some embodiments, the obturator 104 may be replaced with a different elongated medical instrument. As used herein, the term “elongated medical instrument” is a broad term used in its ordinary sense that includes, for example, such devices as needles, cannulas, trocars, obturators, stylets, and the like. Accordingly, the obturator assembly 102 may be referred to more generally as an elongated medical instrument assembly. In like manner, the obturator 104 may be referred to more generally as an elongated medical instrument.

In an embodiment, the obturator assembly 102 includes a coupling hub 103 that is attached to the obturator 104 in any suitable manner (e.g., one or more adhesives or over-molding). The coupling hub 103 can be configured to interface with the driver 101. The coupling hub 103 may alternatively be referred to as an obturator hub 103 or, more generally, as an elongated instrument hub 103.

In an embodiment, the access assembly 109 may further include a shield 105 configured to couple with the obturator 104. The coupling can permit relative longitudinal movement between the obturator 104 and the shield 105, such as sliding, translating, or other movement along an axis of elongation (i.e., axial movement), when the shield 105 is in a first operational mode, and can prevent the same variety of movement when the shield 105 is transitioned to a second operational mode. For example, as further discussed below, the shield 105 may couple with the obturator 104 in a manner that permits longitudinal translation when the obturator 104 maintains the shield 105 in an unlocked state, and when the obturator 104 is moved to a position where it no longer maintains the shield in the unlocked state, the shield 105 may automatically transition to a locked state in which little or no translational movement is permitted between the shield 105 and the obturator 104. Stated otherwise, the shield 105 may be longitudinally locked to a fixed or substantially fixed longitudinal orientation relative to the obturator 104 at which the shield 105 inhibits or prevents inadvertent contact with a distal tip of the obturator. In various embodiments, the shield 105 may be configured to rotate relative to the obturator 104 about a longitudinal axis of the obturator 104 in one or more of the unlocked or locked states. In some embodiments, the shield 105 may be omitted.

With continued reference to FIG. 1A, the needle assembly 162 is referred to as such herein for convenience. In an embodiment, the needle assembly 162 includes a needle 164. However, in various other embodiments, the needle 164 may be replaced with a different instrument, such as, for example, a cannula, a tube, or a sheath, and/or may be referred to by a different name, such as one or more of the foregoing examples. Accordingly, the needle assembly 162 may be referred to more generally as a cannula assembly or as a tube assembly. In like manner, the needle 164 may be referred to more generally as a cannula.

In an embodiment, the needle assembly 162 includes a needle hub 163 that is attached to the needle 164 in any suitable manner. The needle hub 163 can be configured to couple with the obturator hub 103 and may thereby be coupled with the driver 101, as further discussed below. The needle hub 163 may alternatively be referred to as a cannula hub 163.

In an embodiment, the shield 105 is configured to couple with the needle hub 163. The coupling can prevent relative axial or longitudinal movement between the needle hub 163 and the shield 105, such as sliding, translating, or the like, when the shield 105 is in the first operational mode, and can permit the shield 105 to decouple from the needle hub 163 when the shield 105 is transitioned to the second operational mode. For example, as further discussed below, the shield 105 may couple with the needle hub 163 so as to be maintained at a substantially fixed longitudinal position relative thereto when the obturator 104 maintains the shield 105 in the unlocked state, and when the obturator 104 is moved to a position where it no longer maintains the shield in the unlocked state, the shield 105 may automatically transition to a locked state relative to the obturator 104, in which state the shield 105 also decouples from the needle hub 163.

In an embodiment, the shield 105 can be coupled with the obturator 104, the obturator 104 can be inserted into the needle 164, and the obturator hub 103 can be coupled to the needle hub 163 to assemble the access assembly 109. In an embodiment, a cap 107 may be provided to cover at least a distal portion of the needle 164 and the obturator 104 prior to use of the access assembly 109. For example, in an embodiment, a proximal end of the cap 107 can be coupled to the obturator hub 103.

With continued reference to FIG. 1A, the automated driver 108 may take any suitable form. The driver 108 may include a handle 110 that may be gripped by a single hand of a user. The driver 108 may further include an actuator 111 of any suitable variety via which a user may selectively actuate the driver 108 to effect rotation of a coupling interface 112. For example, the actuator 111 may comprise a button, as shown, or a switch or other mechanical or electrical element for actuating the driver 108. In an embodiment, the coupling interface 112 is formed as a socket 113 that defines a cavity 114. The coupling interface 112 can be configured to couple with the obturator hub 103. In an embodiment, the socket 113 includes sidewalls that substantially define a hexagonal cavity into which a hexagonal protrusion of the obturator hub 103 can be received. Other suitable connection interfaces are contemplated.

The automated driver 108 can include an energy source 115 of any suitable variety that is configured to energize the rotational movement of the coupling interface 112. For example, in some embodiments, the energy source 115 may comprise one or more batteries that provide electrical power for the automated driver 108. In other embodiments, the energy source 115 can comprise one or more springs (e.g., a coiled spring) or other biasing member that may store potential mechanical energy that may be released upon actuation of the actuator 111.

The energy source 115 may be coupled with the coupling interface 112 in any suitable manner. For example, in an embodiment, the automated driver 108 includes an electrical, mechanical, or electromechanical coupling 116 to a gear assembly 117. In some embodiments, the coupling 116 may include an electrical motor that generates mechanical movement from electrical energy provided by an electrical energy source 115. In other embodiments, the coupling 116 may include a mechanical linkage that mechanically transfers rotational energy from a mechanical (e.g., spring-based) energy source 115 to the gear assembly 117. The automated driver 108 can include a mechanical coupling 118 of any suitable variety to couple the gear assembly 117 with the coupling interface 112. In other embodiments, the gear assembly 117 may be omitted.

In embodiments, the automated driver 108 can rotate the coupling interface 112, and thereby, can rotate the access assembly 109 at rotational speeds significantly greater than can be achieved by manual rotation of the access assembly 109. For example, in various embodiments, the automated driver 108 can rotate the access assembly 109 at speeds of between 200 and 3,000 rotations per minute. However, greater or lesser rotations per minute are also contemplated.

Further details and embodiments of the intraosseous access system 100 can be found in the patent application publications WO 2018/075694 and WO 2018/165339, each of which is incorporated by reference in its entirety into this application.

With reference to FIGS. 1B and 1C, the obturator assembly 102, which includes the obturator hub 103 and the obturator 104, is shown in greater detail. In the illustrated embodiment, the obturator hub 103 includes a body or housing 120. A proximal end of the housing 120 can be coupled with (e.g., may be attached to or may itself define) a coupling interface 122 for coupling with the coupling interface 112 of the driver 101. In the illustrated embodiment, the coupling interface 122 is formed as a shaft 123 that is configured to be received within the cavity 114 of the socket 113 of the automated driver 108. In particular, the shaft 123 can interface with the socket 113 so as to be rotated thereby. In the illustrated embodiment, the shaft 123 defines a hexagonal cross-section that complements a hexagonal cross-section of the socket 113. Any other suitable arrangement is contemplated. In further embodiments, the socket 113, and the shaft 123 may be reversed, in that the driver 101 may include a shaft and the obturator hub 103 may define a socket for receiving the shaft of the driver 101.

The coupling interface 122 of the obturator hub 103 may further include an annular groove 124 extending around the shaft 123 where the groove 124 is a functional feature of the coupling interface 122. More specifically, the groove 124 is utilized to inhibit separation of the coupling interface 122 from the coupling interface 112 as further described below. In the illustrated embodiment, the coupling interface 122 further includes a retaining member 125 disposed within the groove 124 that, in some embodiments, is utilized in conjunction with the groove 124 to inhibit separation of the coupling interface 122 from the coupling interface 112 as also further described below.

The body or housing 120 may further define a grip 126 that may facilitate manipulation of the obturator hub 103. For example, in the illustrated embodiment, the grip 126 is formed as an indented region of a sidewall 128 that spans a full perimeter of the housing 120.

The illustrated obturator hub 103 includes a skirt 130 that extends distally from a central portion of the housing 120. In the illustrated embodiment, the skirt 130 is defined by a distal portion of the sidewall 128. The skirt 130 can include one or more mechanical coupling members 131 that are configured to selectively couple the obturator hub 103 to the needle hub 163. In the illustrated embodiment, the skirt 130 includes two such mechanical coupling members 131 at opposite sides thereof. In particular, the illustrated embodiment includes two resilient arms or projections 132 that are capable of resiliently deforming in a lateral or radial direction. Each arm can include a snap interface, inward protrusion, or catch 134 at an internal side thereof that can interface with the needle hub 163 to achieve the coupling configuration.

In the illustrated embodiment, the obturator hub 103 further includes a pair of outward protrusions 136 that can assist in coupling the cap 107 to the obturator hub 103. For example, in some embodiments, the cap 107 can define an inner diameter only slightly larger than an outer diameter of the skirt 130. The outward protrusions 136 can slightly deform a proximal end of the cap 107 from a substantially cylindrical shape to a more oblong shape, which may enhance a grip of the cap 107 against the skirt 130. Any other suitable connection arrangement for the cap 107 is contemplated.

With reference to FIG. 1C, the sidewall 128 can further define a coupling interface 137 configured to couple the obturator hub 103 to the needle hub 163 in a manner that causes the obturator hub 103 to rotate in unison with the needle hub 163. In the illustrated embodiment, the coupling interface 137 is formed as a socket 138 into which a shaft portion of the needle hub 163 can be received. The socket 138 can define a keyed shape that permits the obturator hub 103 to be coupled to the needle hub 163 in only one unique rotational or angular orientation. In particular, in the illustrated embodiment, the socket 138 defines an elongated right octagonal prism of which five contiguous sides are substantially identically sized, two enlarged sides that extend from the ends of the five contiguous sides are lengthened relative to the five contiguous sides, and an eighth shorted side that extends between the two enlarged sides is shorter than the five contiguous sides. Any other suitable keying configuration is contemplated. As further discussed below, a keyed interface such as just described can ensure that the obturator 104 and the needle 164 are coupled to each other in a manner that may be desired, in some embodiments, such as to ensure that distal faces of both components are substantially parallel to each other and/or to otherwise ensure that a distal face of the obturator 104 is positioned in a desired manner relative to a distal face of the needle 164. For example, in some embodiments, the keyed interface ensures that the distal faces of the obturator 104 and the needle 164 are substantially parallel to each other and/or ensures that the distal face of the obturator 104 is fully recessed relative to the distal face of the needle 164.

With continued reference to FIG. 1C, in some embodiments, the obturator 104 extends between a proximal end that is coupled to the obturator hub 103 and a distal end 142. The distal end 142 of the obturator 104 has a distal tip 146 at an extremity thereof. In the illustrated embodiment, the housing 120 of the obturator hub 103 substantially encompasses the proximal end 140 of the obturator 104.

The distal end 142 of the obturator 104 includes a distal face 147, which may, in various embodiments, alternatively be referred to as a cut face, ground face, or angled face. In some embodiments, the distal face 147 is formed as a bevel that is at an angle relative to a central longitudinal axis of the obturator 104. For example, in the illustrated embodiment, the distal face 147 defines a substantially planar bevel. In some embodiments, the distal face 147 of the obturator 104 may be configured to be recessed relative to a distal face of the needle 164.

The beveled distal face 147 can be formed in any suitable manner, such as by grinding. For example, the distal face 147 that is substantially planar may be formed by a bias grind (which may also be referred to as a simple bias grind). As further discussed below, in some embodiments, the ground distal face 147 is formed (e.g., ground) at a distal end of a substantially cylindrical rod, and the rod is bent after the distal face 147 has been formed. In other embodiments, the cylindrical rod is bent before the distal face 147 is formed. In still other embodiments, a cylindrical rod is not bent, but rather, each of the distal face 147 and a curved or rounded region 148 adjacent thereto is instead formed by grinding. Other suitable processes for forming the distal end 142 of the obturator 104 are contemplated.

In some embodiments, the obturator 104 may be solid. For example, the obturator 104 may be devoid of passageways or openings extending through any portion thereof. Similarly, the distal end 142 of the obturator 104 may be substantially solid or closed, and may be devoid or openings or passageways therein or therethrough. The distal end 142 of the obturator 104 may substantially fill a lumen of the needle 164, or at least a distal portion of the lumen, to prevent skin or bone from entering into the needle 164 during an insertion event.

The obturator 104 may be formed of any suitable material, such as a substantially rigid material that can resist bending. The material can be sufficiently rigid and strong to inhibit tissue and/or bone from entering a lumen of the needle 164 during an access event. In various embodiments, the obturator 104 can comprise one or more of a rigid plastic or stainless steel. The obturator 104 may, in some instances, provide internal or structural support to the needle 164 during an insertion event. For example, the obturator 104 may act as a stiffener or stylet to inhibit bending of the needle 164 during drilling.

The distal end 142 of the obturator 104 may be shaped and sized to substantially fill a distal end of the needle 164. In various embodiments, such an arrangement can inhibit bending or flattening of the distal end of the needle 164. For example, in some embodiments, there may be a close fit between an inner wall of the distal tip of the needle 164 and an outer surface of the distal end 142 of the obturator 104, and contact between these surfaces can permit the obturator 104 to reinforce the needle 164. For example, in the illustrated embodiment, the distal end 142 of the obturator 104 includes the curved region 148, which may also be referred to as a rounded, bent, or curved region or as a curved surface. A contour of the curved surface 148 can closely match a contour of an inner wall of the needle 164 at the distal end thereof. For example, in various embodiments, these curved surfaces may contact one another along a portion or substantially an entirety of length of the curved surface 148 of the obturator 104 and/or a portion or substantially an entire length of the inner curved surface of the distal end of the needle 164.

In other instances, a small space or gap may be present between the distal end 142 of the obturator 104 and the inner surface of the distal end of the needle 164. In certain of such arrangements, the distal end 142 of the obturator 104 may not initially provide resistance against bending of the needle tip. However, the obturator 104 may instead prevent the needle tip from bending beyond a preset amount. For example, upon bending of the needle tip such that the inner wall comes into contact with the distal end 142 of the obturator 104, the obturator 104 can stop or inhibit further bending of the needle tip.

In the illustrated embodiment, the obturator 104 may further include a recess 150. The recess 150 may be at a position that is between the proximal end and the distal end 142 of the obturator. Stated otherwise, the recess 150 may be positioned proximally relative to the distal tip 146 of the obturator 104. The recess 150 may be of any suitable variety, such as a groove, track, or any other suitable region of indentation or of reduced diameter or reduced thickness, as compared with, for example, a portion of the obturator 104 that is proximal to the recess 150. The recess 150 may or may not extend fully about a longitudinal axis of the obturator 104. In the illustrated embodiment, the recess 150 is defined as a groove 151 that extends fully about the longitudinal axis of the obturator.

FIG. 1D is a side view of a portion of the obturator assembly 102 according to some embodiments disclosed herein. The shaft 123 includes the groove 124. The groove 124 includes width 124B and a bottom surface 124C.

FIG. 1E is a cross section view of the shaft 123 cut along the sectioning line 1E-1E. The retaining member 125 is shown disposed within the groove 124. The retaining member 125 is illustrated as transparent for illustration purposes. The polygonal shape (a hexagon in the illustrated embodiment) includes a number of shaft sides 123C and a corresponding number of the shaft corners 123D. A minor shaft diameter 123B is defined as the distance between opposing shaft sides 123C, and a major shaft diameter 123A is defined as the distance between opposing shaft corners 123D. The groove 124 defines a groove diameter 124A. As illustrated, the groove diameter 124A is less than the minor shaft diameter 123B which is less than the major shaft diameter 123A.

The retaining member 125 defines an outside diameter 125A, an inside diameter 125B, and a cross-sectional diameter 125C in a free state, i.e., absent any deflecting external forces applied thereto. A gap 125D extends between the bottom surface 124C of the groove 124 and an inside surface of the retaining member 125. The gap 125D may be defined to establish desired interface parameters, such as a desired interference fit for example.

The outside diameter 125A of the retaining member 125 is greater than the minor shaft diameter 123B so that a portion of the retaining member 125 extends radially outward of the shaft sides 123C. In the illustrated embodiment, the outside diameter 125A of the retaining member 125 is less than the major shaft diameter 123A which may be advantageous in preventing damage to the retaining member 125 during use, such as insertion of the shaft 123 into socket 113, for example. However, in some embodiments, the outside diameter 125A of the retaining member 125 may be greater than the major shaft diameter 123A.

FIGS. 1F and 1G illustrate a detailed end view of a portion of the driver 101 including the coupling interface 112 and a detailed perspective view of the coupling interface 112, respectively. The polygonal shape (a hexagon in the illustrated embodiment) of the socket 113 includes a number of socket sides 113C and a corresponding number of the socket corners 113D. A minor socket diameter 113B is defined as the distance between opposing socket sides 113C, and a major shaft diameter 113A is defined as the distance between opposing socket corners 113D. The The outside diameter 125A of the retaining member 125 is greater than the minor socket diameter 113B so that a portion of the retaining member 125 contacts the socket sides 113C. In the illustrated embodiment, the outside diameter 125A of the retaining member 125 is less than the major socket diameter 113A which may be advantageous in preventing damage to the retaining member 125 during insertion of the shaft 123 into socket 113. However, in some embodiments, the outside diameter 125A of the retaining member 125 may be greater than the major socket diameter 113A.

In some embodiments, a number (e.g., 1, 2, 3, 4, 5 or 6) of the socket sides 113C may include a recess 113E (e.g., a scallop). Each recess 113E is sized, shaped and positioned to receive a portion of the retaining member 125 therein when the coupling interface 112 of the driver 101 is fully coupled with the coupling interface 122 of the obturator assembly 102, e.g., when the shaft 123 is fully inserted into the socket 113. The disposition of the portion of the retaining member 125 within the recess 113E may establish a greater retention capability over a frictional force alone between the retaining member 125 and the socket sides 113C. In other embodiments, any or all of the socket sides 113C may omit the recess 113E.

FIG. 1H is a cross-sectional side view of the intraosseous access system 100 in an assembled state with the. As shown the coupling interface 122 is coupled with the coupling interface 112. More specifically, the shaft 123 is disposed within the socket 113. The retaining member is disposed within the groove 124 and is in contact with the socket 113.

During use, the user may insert the shaft 123 into the socket 113 where doing so defects the retaining member 125 radially inward (e.g., compresses the retaining member 125 in the groove 124). The compressed retaining member 125 applies a radially outward directed force onto the socker 113 to define a frictional force between the retaining member 125 and the socket 113 so as to inhibit removal of the shaft 123 from the socket 113. In some embodiments described above, the compressed retaining member 125 may expand into one or more recesses 113E within the socket 113 to inhibit separation of the shaft 123 from the socket 113. The frictional force between the retaining member 125 and the socket 113 and/or the expansion of the retaining member 125 into the one or more recesses 113E within the socket 113 may be configured to retain the intraosseous access system 100 in the assembled state during normal use unless a deliberate separating force is applied between the obturator assembly 102 and the driver 101.

In some embodiments, the retaining member 125 may include an O-ring formed of a rubber, a plastic or an elastomeric material, the O-ring defining an unbroken hoop shape, i.e., the retaining member 125 extends continuously and entirely around the hoop shape.

In other embodiments, the retaining member 125 may include a clip, such as an internal circlip, for example. The clip defines a broken shape, i.e., a hoop shape having a gap, such as a C-shape, for example. The clip may be formed of a metal or a plastic material.

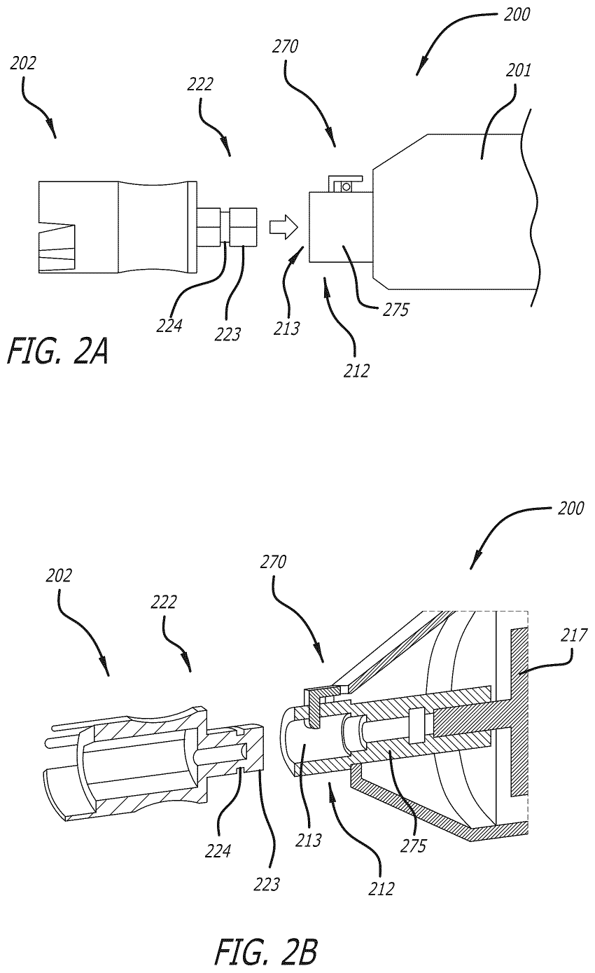

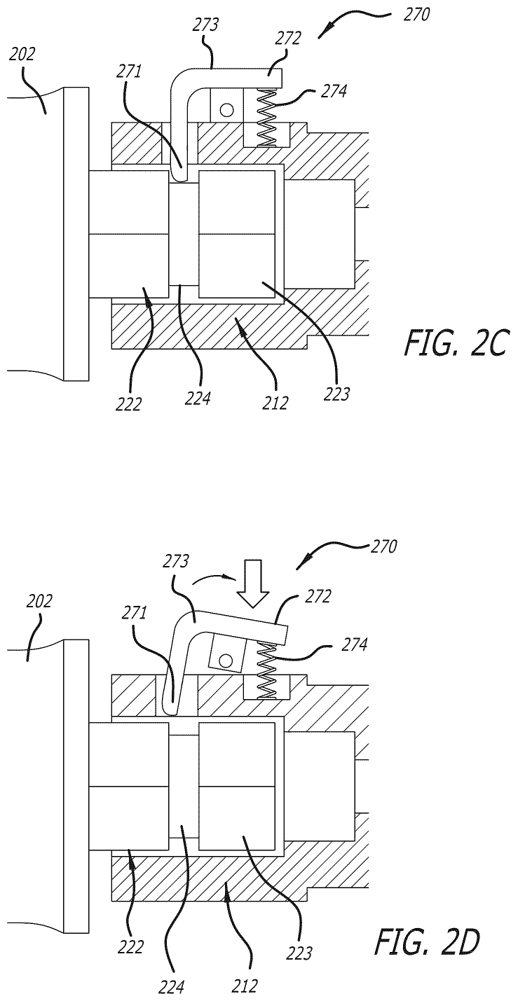

FIGS. 2A-2D illustrate another embodiment of the intraosseous access system 200 that can, in certain respects, resemble components of the intraosseous access system 100 described in connection with FIGS. 1A-1H. It will be appreciated that all the illustrated embodiments may have analogous features. Accordingly, like features are designated with like reference numerals, where the leading digit is increments to “2.” For example a driver of the intraosseous access system 100 is designated by the reference numeral “101” and a driver of the intraosseous access system 200 is designated by the reference numeral “201.” Relevant disclosure set forth above regarding similarly identified features thus may not be repeated hereafter. Moreover, specific features of the intraosseous access system 100 and related components shown in FIGS. 1A-1H may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the intraosseous access system 200 of FIGS. 2A-2D. Any suitable combination of the features, and variations of the same, described with respect to the intraosseous access system 100 and components illustrated in FIGS. 1A-1H can be employed with the intraosseous access system 200 and components of FIGS. 2A-2D and vice versa. This pattern of disclosure applies equally to further embodiments depicted in subsequent figures and described hereafter.

FIGS. 2A and 2B are exploded illustrations of a portion of the intraosseous access system 200 including the obturator assembly 202 and driver 201. The driver 201 includes the coupling interface 212 and the obturator assembly 202 includes the coupling interface 222. The coupling interface 222 includes the shaft 223 having the groove 224. The coupling interface 212 includes the socket 213 which is configured to receive the shaft 223. The coupling interface 212 further includes a socket frame member 275 coupled with the motor 217 such that the socket frame member 275 rotates during operation of the driver 201. The socket frame member 275 may be formed of any suitable material such as a metal or a plastic, for example.

The coupling interface 212 includes a latch mechanism 270 configured to selectively prevent and allow separation of the coupling interface 222 from the coupling interface 212. The latch mechanism 270 is transitionable between a retaining state and a releasing state. In the retaining state, the latch mechanism 270 prevents separation of the coupling interface 222 from the coupling interface 212, and in the releasing state, the latch mechanism 270 allows separation of the coupling interface 222 from the coupling interface 212. The latch mechanism 270 is configured to transition from the retaining state to the releasing state upon actuation of the latch mechanism 270 by the user. In some embodiments, the latch mechanism 270 may be biased toward the retaining state such that the latch mechanism 270 is disposed in the retaining state unless the user actuates the latch mechanism 270 to transition the latch mechanism 270 from the retaining state to the releasing state.

FIGS. 2C and 2D are detailed illustrations of one example of the latch mechanism 270 or more specifically one example of a structure configured to perform the functions of the latch mechanism 270 described above. It is noted that the structure shown and described is only one structure of many structures that could be configured to the perform the functions of the latch mechanism 270 as may be contemplated by one of ordinary skill. As such any and all structures that could be configured to perform the functions are included in this description.

FIG. 2C shows the latch mechanism 270 in the retaining state, and FIG. 2D shows the latch mechanism 270 in the releasing state. The latch mechanism 270 includes an actuation member 272 coupled with a groove engagement member 271. The groove engagement member 271 is disposed within the groove 224 when the latch mechanism 270 is disposed in the retaining state such that the groove engagement member 271 prevents extraction of the shaft 223 from the socket. The actuation member 272 (e.g., a depressible member as shown) is coupled with the groove engagement member 271 via a latch frame 273 such that displacing the actuation member 272 extracts the groove engagement member 271 from the groove 224 to define the releasing state as shown in FIG. 2D. The latch frame 273 is pivotably coupled with the socket frame member 275. A biasing member 274 may be employed to bias the latch frame 273 toward a retaining position consistent with the retaining state of the latch mechanism 270. Of course, the biasing member 274 may take any form such as the compression spring shown, a torsion spring, an extension spring, or a deflectable portion of the latch frame 273 or the socket frame member 275. The latch frame 273 may be formed of any suitable material such as a metal or a plastic, for example.

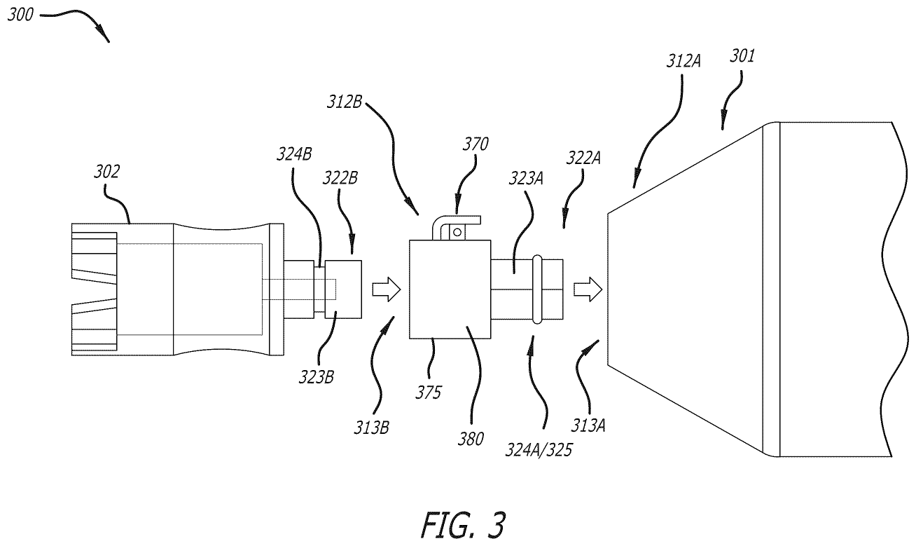

FIG. 3 illustrates another embodiment of an intraosseous access system 300 that can, in certain respects, resemble components of the intraosseous access system 100 described in connection with FIGS. 1A-1H and the intraosseous access system 100 described in connection with FIGS. 2A-2D. The intraosseous access system 300 includes the driver 301, the obturator assembly 302, and an adapter 380. The driver 301 includes a coupling interface 312A that may resemble the components and functionality of the coupling interface 112 of FIGS. 1F and 1G. The adapter 380 includes a coupling interface 322A that may resemble the components and functionality of the coupling interface 122 of FIGS. 1D and 1E. The obturator assembly 302 includes a coupling interface 322B that may resemble the components and functionality of the coupling interface 222 of FIGS. 2A-2D, and the adapter 380 includes a coupling interface 312B that may resemble the components and functionality of the coupling interface 212 FIGS. 2A-2D. The adapter 380 includes the socket frame member 375, the shaft 323A, and the latch mechanism 370. The shaft 323A is configured for insertion into the socket 313A of the driver 301. The adapter 380 further includes the socket 313B configured to receive the shaft 323B of the obturator assembly 302.

In some embodiments, the adapter 380 may be provided as a separate device so that the latching mechanism 370 could be employed with the driver 101. In some instances, the retaining member 125 could be removed from the groove 124 of the obturator assembly 102 (potentially by the user) so that the obturator assembly 102 could be coupled with the adapter 380.

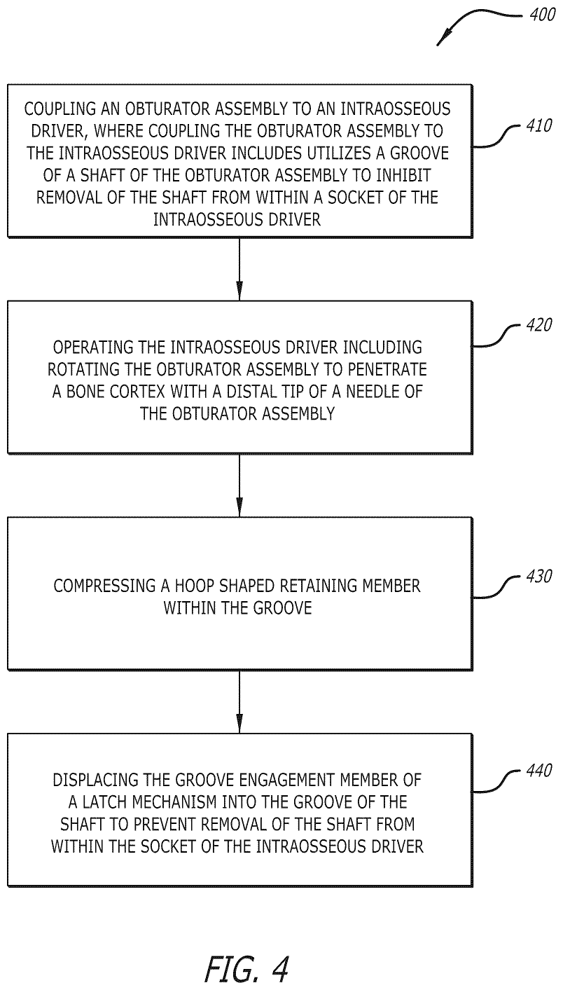

FIG. 4 is a block diagram of a method of accessing a medullary cavity utilizing the systems and/or components thereof described. The method 400 may include all or any subset of the following steps, actions, or processes. The method 400 includes coupling an obturator assembly to an intraosseous driver, where coupling the obturator assembly to the intraosseous driver includes utilizes a groove of a shaft of the obturator assembly to inhibit removal of the shaft from within a socket of the intraosseous driver (block 410). The method 400 further includes operating the intraosseous driver including rotating the obturator assembly to penetrate a bone cortex with a distal tip of a needle of the obturator assembly (block 420). The method 400 may further include compressing a hoop shaped retaining member within the groove (block 430).

In some embodiments of the method 400, the intraosseous driver includes a latch mechanism having a groove engagement member configured for selective displacement into and out of the groove. Accordingly, the method 400 may further include displacing the groove engagement member of a latch mechanism into the groove of the shaft to prevent removal of the shaft from within the socket of the intraosseous driver (block 440).

According to some embodiments of the method disclosed herein, the latch mechanism includes an actuator coupled with the groove engagement member, and the method 400 further includes displacing the actuator to extract the groove engagement member from the groove to enable removal of the shaft from within the socket of the intraosseous driver.

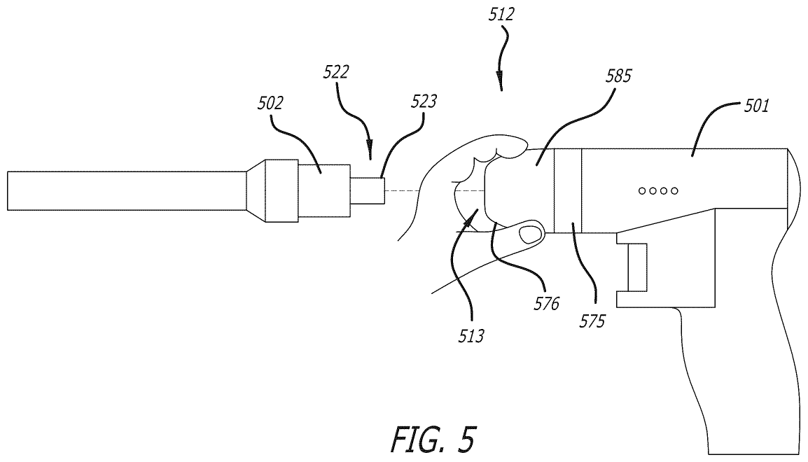

FIG. 5 illustrates another embodiment of an intraosseous access system 500 that can, in certain respects, resemble components of the intraosseous access system 100 described in connection with FIGS. 1A-1H. The intraosseous access system 500 includes the driver 501 and the obturator assembly 502. The driver 501 includes a coupling interface 512 having a socket 513. The coupling interface 512 further includes a chuck 585 coupled with the driver such operation of the driver 501 causes rotation of the chuck 585. The chuck 585 includes socket frame member 575 and a collar 576. The socket 513 is configured to receive the shaft 523 of the coupling interface 522. The chuck 585 is configured to radially clamp onto the shaft 523 when the collar 576 is rotated with respect to the socket frame member 575 to secure the shaft 523 within the socket 513 such that the obturator assembly 502 co-rotates with the chuck 585 during operation of the driver 501.

The chuck 585 may according to any structure configured to perform the functions of the chuck 585 above. It is noted that the structure described below is only one structure of many structures that could be configured to the perform the functions of the chuck 585 as may be contemplated by one of ordinary skill. As such any and all structures that could be configured to perform the functions are included in this description. According to one embodiment, the chuck 585 may include structure similar to a chuck commonly referred to as a Jacob's chuck including a cylindrical sleeve with internal jaws (e.g., three jaws) that can be adjusted radially inward and outward via rotation of collar to grip a shaft securely with respect to the cylindrical sleeve. The shaft 523 may include a polygonal shape or a round shape.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intraosseous access system, comprising:
an intraosseous driver including a driver coupling interface; and
obturator assembly including an obturator coupling interface coupled with the driver coupling interface such that operation of the intraosseous driver causes rotation of the obturator assembly, the obturator coupling interface comprising:
a shaft configured for insertion into a socket of the driver coupling interface so as to cause co-rotation of the shaft and the socket; and
an annular groove extending around the shaft, the annual groove utilized to inhibit decoupling of the obturator coupling interface from the driver coupling interface.

2. The intraosseous access system according to claim 1, wherein the shaft includes:
a polygonal shape including a number of shaft sides and a corresponding number of shaft corners, the shaft corners defining a major diameter of the shaft and the shaft sides defining a minor diameter of the shaft that is less than the major diameter of the shaft; and
a groove diameter less than the minor diameter of the shaft.

3. The intraosseous access system according to claim 2, wherein:
the socket includes a polygonal shape that corresponds to the polygonal shape of the shaft,
the socket includes a number of shaft sides and a corresponding number of shaft corners,
socket corners define a major diameter of the socket, and
socket sides define a minor diameter of the socket that is less than the major diameter of the socket.

4. The intraosseous access system according to claim 3, wherein the obturator coupling interface further comprises a retaining member disposed within the groove, the retaining member defining, in a free state, a hoop shape having an outside diameter and an inside diameter.

5. The intraosseous access system according to claim 4, wherein the inside diameter is less than the minor diameter of the shaft and the outside diameter is greater than the minor diameter of the shaft.

6. The intraosseous access system according to claim 4, wherein the outside diameter is less than the major diameter of the shaft.

7. The intraosseous access system according to claim 4, wherein the outside diameter is greater than the minor diameter of the socket such that, during insertion of the shaft into the socket, the socket sides deflect the retaining member radially inward.

8. The intraosseous access system according to claim 4, wherein the outside diameter is less than the major diameter of the socket.

9. The intraosseous access system according to claim 4, wherein the retaining member defines a frictional force with the socket sides, the frictional force inhibiting removal of the shaft from the socket.

10. The intraosseous access system according to claim 9, wherein the minor diameter of the socket, the groove diameter, the outside diameter and the inside diameter are sized to define an interference fit of the retaining member between the socket sides and a bottom surface of the groove, the interference fit defining the frictional force.

11. The intraosseous access system according to claim 10, wherein at least a subset of the socket sides includes a recess configured to receive a portion of the retaining member therein so as to inhibit separation of the shaft from the socket.

12. The intraosseous access system according to claim 4, wherein retaining member extends continuously and entirely around the hoop shape.

13. The intraosseous access system according to claim 12, wherein the retaining member includes an O-ring formed of a plastic or an elastomeric material.

14. The intraosseous access system according to claim 1, wherein the driver coupling interface includes a latch mechanism configured to transition between:
a retaining state, wherein separation of the obturator coupling interface from the driver coupling interface is prevented, and
a releasing state, wherein separation of the obturator coupling interface from the driver coupling interface is allowed.

15. The intraosseous access system according to claim 14, wherein the latch mechanism includes an actuation member coupled with a groove engagement member such that displacing the actuation member extracts the groove engagement member from the groove to transition the latch mechanism from the retaining state to the releasing state.

16. The intraosseous access system according to claim 1, further including an adapter configured for placement between the obturator assembly and intraosseous driver, wherein the adapter includes:
an adapter shaft configured for insertion into the socket of the intraosseous driver; and
an adapter socket configured to receive the shaft of the obturator assembly.

17. An adapter for an intraosseous access system, wherein the adapter includes an adapter frame having:
an adapter socket configured to receive a shaft of an obturator assembly;
an adapter shaft configured for insertion into a socket of an intraosseous driver; and
a latch mechanism, including a groove engagement member configured for selective displacement into and out of a groove of the shaft of the obturator assembly.

18. A method of accessing a medullary cavity, comprising:
coupling an obturator assembly to an intraosseous driver, wherein coupling the obturator assembly to the intraosseous driver includes utilizing a groove of a shaft of the obturator assembly to inhibit removal of the shaft from within a socket of the intraosseous driver; and
operating the intraosseous driver including rotating the obturator assembly to penetrate a bone cortex with a distal tip of a needle of the obturator assembly.

19. The method according to claim 18, wherein utilizing the groove of the shaft includes compressing a hoop shaped retaining member within the groove.

20. The method according to claim 18, wherein:
the intraosseous driver includes a latch mechanism including a groove engagement member configured for selective displacement into and out of the groove, and
coupling the obturator assembly to the intraosseous driver includes displacing the groove engagement member into the groove of the shaft to prevent removal of the shaft from within the socket of the intraosseous driver.

21. The method according to claim 20, wherein the latch mechanism includes an actuator coupled with the groove engagement member, the method further comprising displacing the actuator to extract the groove engagement member from the groove to enable removal of the shaft from within the socket of the intraosseous driver.

22. An intraosseous access system, comprising:
an obturator assembly including an obturator coupling interface having a shaft; and
an intraosseous driver including a driver coupling interface having a chuck, wherein the chuck includes:
a socket frame member defining a socket configured to receive the shaft; and
a collar rotatable with respect to the socket frame member, wherein rotation of the collar with respect to the socket frame member causes the chuck to radially clamp onto the shaft such that:
the shaft and chuck co-rotate, and
removal of the shaft from the chuck is prevented.
